(19) United States
(12) Patent Application Publication
(10) Pub. No.: US 2023/0139235 A1
(43) Pub. Date: May 4, 2023

(54) Head-Worn Oxygen Tube Device

(71) Applicants: George Griffith, Cortez, CO (US); Cindy Sutherland, Cortez, CO (US)

(72) Inventors: George Griffith, Cortez, CO (US); Cindy Sutherland, Cortez, CO (US)

(21) Appl. No.: 18/049,392

(22) Filed: Oct. 25, 2022

Related U.S. Application Data
(60) Provisional application No. 63/273,282, filed on Oct. 29, 2021.

(51) Int. Cl. A61M 16/06

(52) U.S. Cl. CPC A61M 16/0683

(57) ABSTRACT

The present invention relates to a head-worn oxygen tube device that can be used in conjunction with head-worn oxygen tubes or other head-worn masks. The device is primarily comprised of a body with a first end having at least one fastener and a second end having at least one fastener. The device can be worn on the head of the user such that each oxygen tube can be secured by the fastener. Due to the length of the body, each fastener sits above each ear of the user such that each oxygen tube no longer rests on the ears of the user. However, the device still allows a nasal cannula to be worn and used.

### CROSS-REFERENCE TO RELATED APPLICATION

The present application claims priority to, and the benefit of, U.S. Provisional Application No. 63/273,282, which was filed on Oct. 29, 2021, and is incorporated herein by reference in its entirety.

### FIELD OF THE INVENTION

The present invention relates generally to the field of oxygen tubes. More specifically, the present invention relates to a head-worn oxygen tube device. The device is primarily comprised of a body with a first end having at least one fastener and a second end having at least one fastener. The device can be worn on the head of the user such that each oxygen tube can be secured by the fastener. As a result of the length of the body, each fastener sits above each ear of the user such that each oxygen tube no longer rests on the ears of the user. However, the device still allows a nasal cannula to be worn and used. Accordingly, the present disclosure makes specific reference thereto. Nonetheless, it is to be appreciated that aspects of the present invention are also equally applicable to other like applications, devices, and methods of manufacture.

### BACKGROUND

Many individuals are required to wear oxygen tubes for various health conditions. While worn, oxygen tubes often rest on the ears of the wearer to keep the oxygen cannula supported below the nose of the wear. However, oxygen tubes resting on a wearer's ears can cause hearing aids to malfunction. In addition, oxygen tubes resting on a wearer's ears can cause sores and discomfort to occur over time.

Therefore, there exists a long-felt need in the art for a device that can be used to improve the wearing experience of oxygen tubes. There also exists a long-felt need in the art for a head-worn oxygen tube device that allows a user to comfortably wear oxygen tubes. More specifically, there exists a long-felt need in the art for a head-worn oxygen tube device that prevents discomfort by preventing oxygen tubes from resting on the ears of a user. In addition, there exists a long-felt need in the art for a head-worn oxygen tube device that prevents hearing aid malfunctions by preventing oxygen tubes from resting on the ears of a user.

The subject matter disclosed and claimed herein, in one embodiment thereof, comprises a head-worn oxygen tube device. The device is primarily comprised of a body with a first end having at least one fastener and a second end having at least one fastener. The device can be worn on the head of the user such that each oxygen tube can be secured by the fastener. Due to the length of the body, each fastener sits above each ear of the user. As a result, each oxygen tube no longer rests on the ears of the user, while still allowing a nasal cannula to be worn and used.

In this manner, the head-worn oxygen tube device of the present invention accomplishes all of the forgoing objectives and provides a device that can be used to improve the wearing experience of oxygen tubes. The device accomplishes this by reducing discomfort by preventing oxygen tubes from resting on the ears of a user. In addition, the device prevents hearing aid malfunctions by preventing oxygen tubes from resting on the ears of a user.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a head-worn oxygen tube device. The device is primarily comprised of a body with a first end having at least one fastener and a second end having at least one fastener. The body of the device is preferably made from a fabric material with elastic properties such that it can comfortably fit and stretch to the head of a user. In a differing embodiment, the body is made from a non-elastic fabric material having at least one body fastener such as, but not limited to, a buckle that allows a user to lengthen and shorten the overall length of the body based on the head size of the user.

In one embodiment, at least one sleeve may attach around the body via at least one opening that allows the body to be fed through the sleeve. The sleeve may be comprised of at least one gel that can be heated or frozen to provide relief to the head of the user while the device is worn. The sleeve and/or body may also be comprised of a padding for additional comfort.

In one embodiment, the first end and second end of the body are comprised of at least one attachment member that allows at least one fastener to removably attach to the first end and the second end. The attachment members may be, but are not limited to, a loop, a clip, a ring, etc. In another embodiment, the fasteners are fixedly attached to the first end and second end. The fasteners of each end engage one side of an oxygen tube such that the tube rests on each fastener above the ear of the user. The fasteners may be, but are not limited to, a u-shaped or J-shaped hook, a clip, a carabiner, a clasp, a tie, a set of jaws, etc.

The device is also comprised of a method of use. First, a user places the body on their head. Then, the fastener of the first end is attached to one oxygen tube on one side of the user's head. Next, the fastener of the second end is attached to the other oxygen tube on the other side of the user's head.

Accordingly, the head-worn oxygen tube device of the present invention is particularly advantageous as it can be used to improve the wearing experience of oxygen tubes by reducing discomfort by preventing oxygen tubes from resting on the ears of a user. In addition, the device prevents hearing aid malfunctions by preventing oxygen tubes from resting on the ears of a user. In this manner, the head-worn oxygen tube device overcomes the disadvantages of existing head-worn oxygen tubes known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a head-worn oxygen tube device of the present invention in accordance with the disclosed architecture;

FIG. 2 illustrates a perspective view of one potential embodiment of a head-worn oxygen tube device of the present invention in accordance with the disclosed architecture;

FIG. 3 illustrates a perspective view of one potential embodiment of a head-worn oxygen tube device of the present invention while being worn on a head of a user also wearing an oxygen tube in accordance with the disclosed architecture; and

FIG. 4 illustrates a flowchart of a method of using one potential embodiment of a head-worn oxygen tube device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that can be used to improve the wearing experience of oxygen tubes. There also exists a long-felt need in the art for a head-worn oxygen tube device that allows a user to comfortably wear oxygen tubes. More specifically, there exists a long-felt need in the art for a head-worn oxygen tube device that prevents discomfort by preventing oxygen tubes from resting on the ears of a user. In addition, there exists a long-felt need in the art for a head-worn oxygen tube device that prevents hearing aid malfunctions by preventing oxygen tubes from resting on the ears of a user.

The present invention, in one exemplary embodiment, is comprised of a head-worn oxygen tube device primarily comprised of a body with a first end having at least one fastener and a second end having at least one fastener. The body of the device is preferably made from a fabric material with elastic properties such that it can comfortably fit and stretch to the head of a user. In a different embodiment, the body is made from a non-elastic fabric material. In this embodiment, the body has at least one body fastener such as, but not limited to, a buckle that allows a user to lengthen and shorten the overall length of the body based on the head size of the user.

At least one sleeve may attach around the body via at least one opening that allows the body to be fed through the sleeve. The sleeve may be comprised of at least one gel. As a result, the sleeve can be heated or frozen to provide relief to the head of the user while the device is worn. The sleeve and/or body may also be comprised of a padding for additional comfort.

In one embodiment, the first end and second end of the body are comprised of at least one attachment member. The attachment members allow at least one fastener to removably attach to both ends. The attachment members may be, but are not limited to, a loop, a clip, a ring, etc. In another embodiment, the fasteners are fixedly attached to the first end and second end. The fasteners of each end engage one side of an oxygen tube such that the tube rests on each fastener above the ear of the user. The fasteners may be, but are not limited to, a u-shaped or J-shaped hook, a clip, a carabiner, a clasp, a tie, a set of jaws, etc.

The device is also comprised of a method of use. First, a user places the body on their head. Then, the fastener of the first end is attached to one oxygen tube on one side of the user's head. Next, the fastener of the second end is attached to the other oxygen tube on the other side of the user's head.

Accordingly, the head-worn oxygen tube device of the present invention is particularly advantageous as it can be used to improve the wearing experience of oxygen tubes by reducing discomfort by preventing oxygen tubes from resting on the ears of a user. In addition, the device prevents hearing aid malfunctions by preventing oxygen tubes from resting on the ears of a user. In this manner, the head-worn oxygen tube device overcomes the disadvantages of existing head-worn oxygen tubes known in the art.

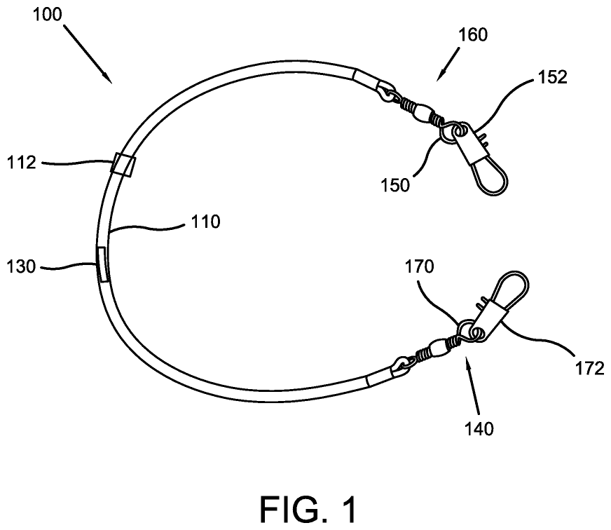

Referring initially to the drawings, FIG. 1 illustrates a perspective view of one potential embodiment of a head-worn oxygen tube device 100 of the present invention in accordance with the disclosed architecture. The device 100 is primarily comprised of a body 110 with a first end 140 having at least one fastener 152 and a second end 160 having at least one fastener 172. The body 110 of the device 100 is preferably made from a fabric material with elastic properties. In this manner, the body 110 can comfortably fit and stretch to the head 10 of a user. Said material includes, but is not limited to, spandex, knit, rubber, latex, neoprene rubber, elastane, etc. In a differing embodiment, the body 110 is made from a non-elastic fabric material. In this embodiment, the body 110 is comprised of at least one body fastener 112. The body fastener 112 allows a user to lengthen and shorten the overall length of the body 110 based on the head 10 size of the user. The fastener 112 may be any fastener type known in the art but is preferably a sliding buckle or another similar fastener of the like. In one embodiment, the body 110 is made from a leather material.

In one embodiment, at least one sleeve 120 may attach around the body 110. In one embodiment, the sleeve 120 has at least one opening 122 that allows the body 110 to be fed through the sleeve. In another embodiment, the sleeve 120 may have at least one fastener 124 that attaches to at least one reciprocating fastener 114 located on the body 110. The fasteners 114,124 may be any fastener type known in the art such as, but not limited to, hook and loop, magnet, adhesive, snap button, etc. The sleeve 120 is preferably made from a fabric material such as, but not limited to, vinyl, canvas, cashmere, chenille, chiffon, cotton, damask, jersey, lace, linen, wool, modal, polyester, satin, silk, spandex, suede, tweed, twill, velvet, acrylic, modacrylic, nylon, polypropylene, polyurethane, polyvinyl chloride, polyethylene, vinylidene, benzoate, aramid, rayon, acetate, triacetate, etc.

In one embodiment, the sleeve 120 is comprised of at least one gel 126. The gel 126 may be heated or frozen to provide relief to the head 10 of the user while the device 100 is worn. The gel 126 may be any type of reusable gel that can be heated and/or cooled. In one embodiment, the sleeve 120 or body 110 is comprised of a padding 130. The padding 130 adds extra comfort to the sleeve 120 or body 110. The padding 130 may be any type known in the art such as, but not limited to, foam, memory foam, etc.

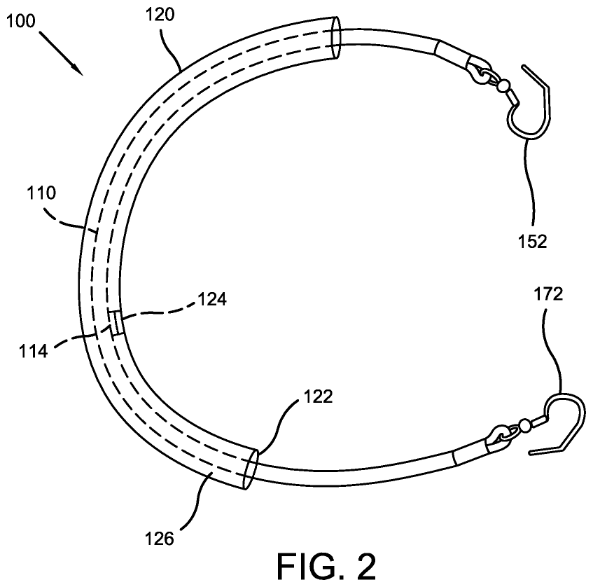

In one embodiment, the first end 140 of the body 110 is comprised of at least one attachment member 150 that allows at least one fastener 152 to removably attach to the first end 140. The attachment member 150 may be, but is not limited to, a loop, a clip, a ring, etc. In another embodiment, the fastener 152 is fixedly attached to the first end 140. The fastener 152 engages one side of an oxygen tube 12 such that the tube 12 rests on the fastener 152 instead of on the ear 11 of the user. It should be appreciated that the length of the body 110 allows the fastener 152 to sit above the ear 11 of the user. The fastener 152 may be any fastener known in the art. In one embodiment, the fastener 152 is comprised of a u-shaped or J-shaped hook, as seen in FIG. 2. In another embodiment, the fastener 152 is comprised of a clip, a carabiner, a clasp, a tie, a set of jaws, etc.

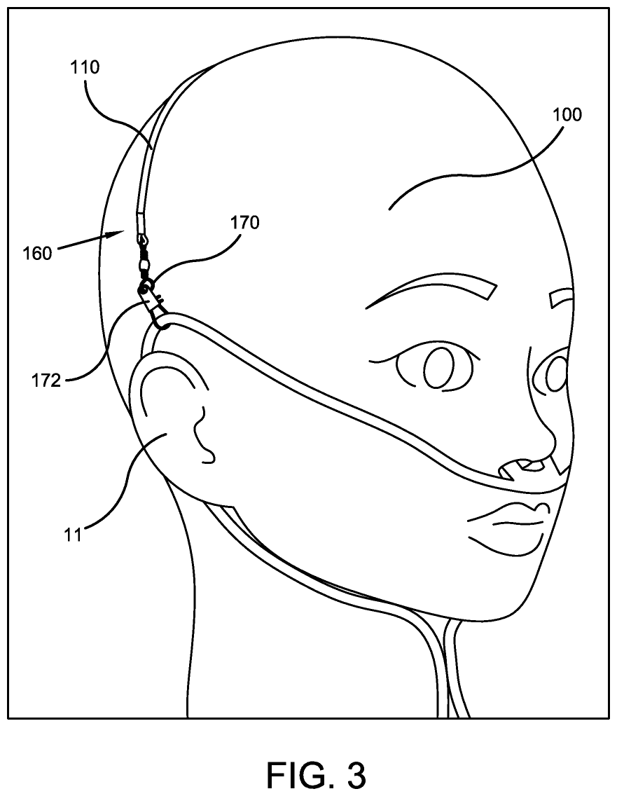

The second end 160 of the body 110 is comprised of at least one attachment member 170 that allows at least one fastener 172 to removably attach to the second end 160. The attachment member 170 may be, but is not limited to, a loop, a clip, a ring, etc. In another embodiment, the fastener 172 is fixedly attached to the second end 160. The fastener 172 engages one side of an oxygen tube 12 such that the tube 12 rests on the fastener 172 instead of on the ear 11 of the user. It should be appreciated that the length of the body 110 allows the fastener 172 to sit above the ear 11 of the user, as seen in FIG. 3. The fastener 172 may be any fastener known in the art. In one embodiment, the fastener 172 is comprised of a u-shaped or J-shaped hook. In another embodiment, the fastener 172 is comprised of a clip, a carabiner, a clasp, a tie, a set of jaws, etc.

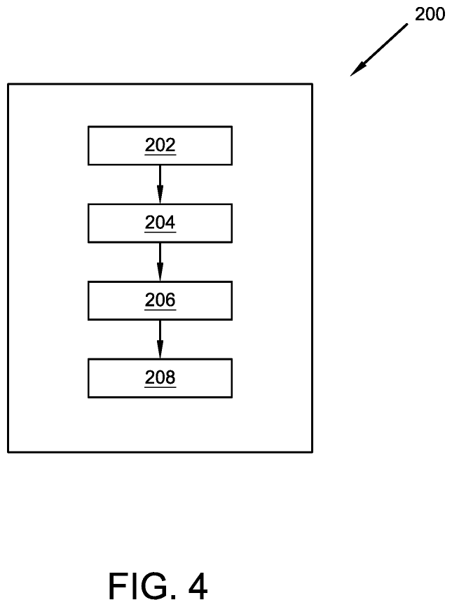

FIG. 4 illustrates a flowchart of a method 200 of using one potential embodiment of a head-worn oxygen tube device 100 of the present invention in accordance with the disclosed architecture. The device 100 is also comprised of a method of use 200. First, a user places the body 110 on their head 10 [Step 202]. In an embodiment with a length adjustable body 110, the body 110 can be adjusted to the desired length [Step 204]. Next, the fastener 152 of the first end 140 is attached to one oxygen tube 12 on one side of the user's head 10 [Step 206]. Then, the fastener 172 of the second end 160 is attached to the other oxygen tube 12 on the other side of the user's head 10 [Step 208].

It should be appreciated that the device 100 can be used with any type of face and ear-worn mask known in the art and is not limited to only oxygen tubes 12.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “head-worn oxygen tube device” and “device” are interchangeable and refer to the head-worn oxygen tube device 100 of the present invention.

Notwithstanding the foregoing, the head-worn oxygen tube device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the head-worn oxygen tube device 100 as shown in the FIGS. are for illustrative purposes only, and that many other sizes and shapes of the head-worn oxygen tube device 100 are well within the scope of the present disclosure. Although the dimensions of the head-worn oxygen tube device 100 are important design parameters for user convenience, the head-worn oxygen tube device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A head-worn oxygen tube device comprising:
a body;
a first end;
a first fastener positioned on the first end;
a second end; and
a second fastener positioned on the second end.

2. The head-worn oxygen tube device of claim 1, wherein the body is comprised of a leather material.

3. The head-worn oxygen tube device of claim 1 further comprising a buckle, wherein the body can adjust in length via the buckle.

4. The head-worn oxygen tube device of claim 1, wherein the body is comprised of a padding.

5. The head-worn oxygen tube device of claim 1, wherein the first fastener is comprised of a u-shaped hook or a j-shaped hook.

6. The head-worn oxygen tube device of claim 1, wherein the second fastener is comprised of a u-shaped hook or a j-shaped hook.

7. A head-worn oxygen tube device comprising:
a body;
a sleeve;
a first end;
a first fastener;
a second end; and
a second fastener.

8. The head-worn oxygen tube device of claim 7, wherein the body is comprised of a body fastener.

9. The head-worn oxygen tube device of claim 8, wherein the sleeve is positioned around a body.

10. The head-worn oxygen tube device of claim 9, wherein the sleeve is comprised of a sleeve fastener that engages the body fastener.

11. The head-worn oxygen tube device of claim 7, wherein the sleeve is comprised of a gel.

12. The head-worn oxygen tube device of claim 7, wherein the body is comprised of an elastic material.

13. The head-worn oxygen tube device of claim 7, wherein the sleeve is comprised of a padding.

14. The head-worn oxygen tube device of claim 7, wherein the first fastener is comprised of a clip, a carabiner, or a clasp.

15. The head-worn oxygen tube device of claim 14, wherein the first fastener attaches to a first attachment member of the body.

16. The head-worn oxygen tube device of claim 15, wherein the first attachment member is comprised of a loop or a ring.

17. The head-worn oxygen tube device of claim 7, wherein the second fastener is comprised of a clip, a carabiner, or a clasp.

18. The head-worn oxygen tube device of claim 17, wherein the second fastener attaches to a second attachment member of the body.

19. The head-worn oxygen tube device of claim 18, wherein the second attachment member is comprised of a loop or a ring.

20. A method of using a head-worn oxygen tube device, the method comprising the steps of:
placing a body of a head-worn oxygen tube device on a head of a user;
placing a first oxygen tube on a first fastener of the head-worn oxygen tube device; and
placing a second oxygen tube on a second fastener of the head-worn oxygen tube device.
